(19) United States
(12) Patent Application Publication
(10) Pub. No.: US 2026/0083646 A1
(43) Pub. Date: Mar. 26, 2026

(54) METHOD FOR FORMING A DISPERSION COMPRISING DROPS AND ASSOCIATED DEVICE

(71) Applicant: CAPSUM, MARSEILLE (FR)

(72) Inventors: Duncan GILBERT, LA CRAU (FR); Mathieu GOUTAYER, Saint-Malo (FR)

(21) Appl. No.: 19/111,062

(22) PCT Filed: Sep. 13, 2023
(86) PCT No.: PCT/EP2023/075101

§ 371 Date: Mar. 12, 2025

(30) Foreign Application Priority Data
Sep. 13, 2022 (FR) FR2209176

(51) Int. Cl. A61K 8/06; A61K 8/04; A61Q 19/00; B01F 23/41; B01F 23/451; B01F 25/51; B01F 101/21

(52) U.S. Cl. CPC A61K 8/062; A61K 8/042; A61Q 19/00; B01F 23/4145; B01F 23/451; B01F 25/51; A61K 2800/10; A61K 2800/30; A61K 2800/33; A61K 2800/34; B01F 2101/21; B01F 2215/0431; B01F 2215/044

(57) ABSTRACT

A method of forming a dispersion comprising drops having a fatty phase dispersed in a gelled continuous aqueous phase. The method includes: providing a fatty phase comprising at least one oil; providing an aqueous phase, that is substantially immiscible with the fatty phase, comprising at least water and at least one hydrophilic gelling agent capable of gelling in the presence of at least one salt; forming fatty phase drops in the aqueous phase or the gelled continuous aqueous phase; conveying the drops in a circulation duct; recovering a dispersion comprising drops and the gelled continuous aqueous phase in a container. The method further includes: before the forming step, adding, to the aqueous phase, at least one portion of an aqueous solution; and/or injecting at least one portion of the aqueous solution into the circulation duct or at the outlet of the circulation duct upstream of the container.

The present invention relates to a method for forming a stable oil-in-water dispersion comprising a gelled continuous aqueous phase comprising at least one hydrophilic gelling agent apt for gelling in the presence of at least one salt and drops, in particular macroscopic drops, of a dispersed fatty phase.

To date, stable dispersions of drops, in particular macroscopic drops exist, of a fatty phase dispersed in a continuous aqueous phase, obtained by means of a microfluidic method, described in particular in WO2017046305.

Such microfluidic methods are particularly effective for forming stable dispersions comprising drops of perfectly controlled size and exhibiting satisfactory properties in terms of transparency, texture, viscosity and suspension of the drops of the fatty phase dispersed in the continuous aqueous phase.

Such microfluidic methods are particularly sensitive and many parameters can alter the robustness thereof and/or the kinetic stability of the dispersions obtained. More particularly, the aqueous phase must be sufficiently fluid and homogeneous during the emulsification step and sufficiently viscous once the dispersion is formed, so as to produce the suspension of the drops in the continuous aqueous phase.

The implementation and robustness of the microfluidic methods, as well as the satisfactory properties mentioned hereinabove, depend in particular on the presence, in a continuous aqueous phase, of a pH-dependent gelling agent, generally such as a carbomer (or acrylic polymer), e.g. same marketed by Lubrizol under the name Carbopol. The gelling/suspending effect of the carbomers is activated after the formation of the dispersion by the “neutralization” of the aqueous phase, by addition of a sodium hydroxide solution. Thereby, before the formation of the dispersion, the aqueous phase is provided with an acidic pH, namely comprised between 3.5 and 5.5, preferably between 4 and 5, in order to guarantee a fluidity compatible with the microfluidic method. After the formation of the dispersion, the gelling of the aqueous phase is carried out by means of a step of injection of the sodium hydroxide solution. Such a method is described in the application WO2015055748.

However, carbomers are synthetic hydrophilic polymers of acrylic acid, and are hence of petrochemical origin. The use thereof in cosmetics is increasingly controversial. Moreover, the European Chemicals Agency (ECHA) is studying, in coordination with the REACH Committee, a ban on microplastics in cosmetics, where carbomers are present.

Given the foregoing, the presence of carbomers is nowadays imperative for guaranteeing the manufacture by microfluidics of stable dispersions, in particular macroscopic dispersions. The replacement of carbomers thus becomes an important and critical problem, in particular in the case of a sudden change of vision of the consumers regarding carbomers, the integration of the raw materials by customers in their blacklist, or even the prohibition of the uses thereof in cosmetics by the applicable regulations.

In addition, carbomers, by interfacial complex coacervation reaction with a lipophilic cationic polymer present in the fatty phase, in particular amodimethicone, can also be involved in the formation of a shell. The fine and non-residual shell upon application makes it possible to impart macroscopic dispersions obtained by a microfluidic method with a better mechanical resistance.

Many attempts to substitute carbomers with natural hydrophilic gelling agents have been envisaged. However, such substitution is difficult because of the lower performances of the natural hydrophilic gelling agents in terms, in particular, of transparency and non-stickiness.

While many natural hydrophilic gelling agents are sensitive to temperature, few of same have the property of modulating the viscosity thereof upwards according to a physical-chemical parameter at room temperature, like carbomers.

Moreover, as soon as it is sought to obtain a gelled aqueous phase with a certain viscosity, gels based on natural hydrophilic gelling agent(s), in particular apt to gel in the presence of at least one salt, are very often firm and brittle, where gels based on carbomer(s) are flexible, fluid and slightly cohesive. “Slightly cohesive” refers to a gel having an adjusted compromise between the ability to stick to itself, unlike a brittle gel, without the property being too exacerbated, otherwise one would be faced with a gel having a texture/behavior such as an “egg white”, which is undesirable.

There is thus a need for new dispersions comprising droplets of a fatty phase, in particular of macroscopic size, of a fatty phase dispersed in a continuous aqueous phase and which remain satisfactory in terms of kinetic stability, transparency, texture, feeling, and comfort upon application, despite the absence of amodimethicone, or of shell.

There is also a need for new gelled aqueous phase formulations with variable viscosity, without carbomer, and which, after activation, remain flexible, fluid and slightly cohesive, even at high viscosity.

A goal of the invention is thus to provide a simple microfluidic manufacturing method for a dispersion containing drops, in particular macroscopic drops, of a fatty phase in stable suspension in a continuous aqueous phase providing a non-petrochemical and non-microplastic alternative to carbomers.

Thereby, the subject matter of the present invention relates to a method of forming (or manufacturing) a dispersion (10) comprising drops (12) having a fatty phase (14), which drops being dispersed in a gelled continuous aqueous phase (22), the method comprising the steps of:
(i) providing a fatty phase (14) comprising at least one oil and, optionally, at least one lipophilic gelling agent that is preferably heat-sensitive
(ii) providing an aqueous phase (16), that is substantially immiscible with the fatty phase (14), comprising at least water and at least one hydrophilic gelling agent capable of gelling in the presence of at least one salt;
(iii) forming fatty phase drops (14) in the aqueous phase (16) or the gelled continuous aqueous phase (22);
(iv) flow, in a circulation duct (38), of the drops (12);
(v) recovering a dispersion (10) comprising drops (12) and the gelled continuous aqueous phase (22) in a container (33);
characterized in that the method comprises at least one of the steps (vi) of:
(vi1) before step (iii), adding, to the aqueous phase (16), at least one portion of an aqueous solution (62); and/or
(vi2) injecting at least one portion of the aqueous solution (62) into the circulation duct (38) or at the outlet of the circulation duct (38) upstream of the container (33),
the aqueous solution (62) comprising at least one salt capable of reacting with the hydrophilic gelling.

Given the foregoing, the aqueous phase (16) is advantageously free of carbomer.

Also, there is an increasing demand from consumers for cosmetic compositions free of silicone compounds because of the environmental impact thereof, since same are not biodegradable, and/or due to the suspected health-risk same carry.

Thereby, a dispersion according to the invention, and more particularly the fatty phase (14), is advantageously free of amodimethicone.

A method according to the invention, when step (vi) is represented entirely or partially by step (vi1), may be indifferently designated in the remainder of the description by “first method”.

A method according to the invention, when step (vi) is represented entirely or partially by step (vi2), may be indifferently referred to hereinafter in the description by “second method”.

In the remainder of the description, the solution comprising at least one salt may be referred to without distinction by the expression “solution (62)”, “aqueous solution (62)”, “additional solution” or “BF”.

As illustrated by the examples, a microfluidic method according to the invention is advantageous in that same is used to manufacture stable dispersions, simple or multiple, comprising drops of fatty phase, of controlled or even macroscopic size, and endowed with satisfactory optical properties without resorting to the use of any carbomer, in a continuous, simple and robust manner.

A method according to the invention is also advantageous in that same is based on a microfluidic alternative:
non-microplastic;
using an adjustable or “scalable” viscosity of the continuous aqueous phase via a pair of raw materials of natural origin;
   compatible with temperatures up to 50° C.; and
   apt to impart satisfactory properties to the continuous phase in terms of transparency, texture and sensory experience.

Adjustable or “scalable” viscosity refers to a viscosity compatible with the constraints of a microfluidic method until the dispersion is formed under normal conditions, said viscosity then being able to be increased so as to produce a satisfactory suspension of the drops of fatty phase in the continuous aqueous phase.

Also, such alternative is advantageous in that same makes it possible to access dispersions comprising a continuous aqueous phase endowed with properties which are particularly satisfactory in terms of kinetic stability, more particularly suspension-ability of the drops, stickiness, and comfort during application (more particularly play-time). And yet, such combination of criteria is a compromise that is not obvious, in particular given the absence of carbomer or of a shell.

Furthermore, the present invention makes it possible to obtain a dispersion, in particular a cosmetic dispersion, comprising at least one fluid gelled aqueous phase (22) comprising at least one salt and at least one hydrophilic gelling agent apt to gel in the presence of the salt(s), the dispersion being free of carbomer and optionally of amodimethicone.

“Fluid”, as defined by the present invention, refers to a gelled aqueous phase which, at ambient temperature and at atmospheric pressure, retains the capacity thereof to flow under its own weight. More particularly, a gelled aqueous phase according to the invention retains the ability to take the shape of the container thereof. In other words, a gelled aqueous phase according to the invention is not in the form of a solid block, and in particular is not in the form of a firm and brittle solid gel.

Against all expectations, the inventors observed that the invention makes it possible to obtain fluid (or liquid) gels with properties similar to the properties observed with carbomer gels, in particular in terms of transparency and non-stickiness.

Furthermore, the invention makes it possible to obtain suspension liquid gels, which is used for the manufacture of dispersions. As such, and unexpectedly, a gelled aqueous phase (22) according to the invention remains compatible with a microfluidic method at ambient temperature, without prejudice to the above-mentioned advantages in terms of transparency and non-stickiness.

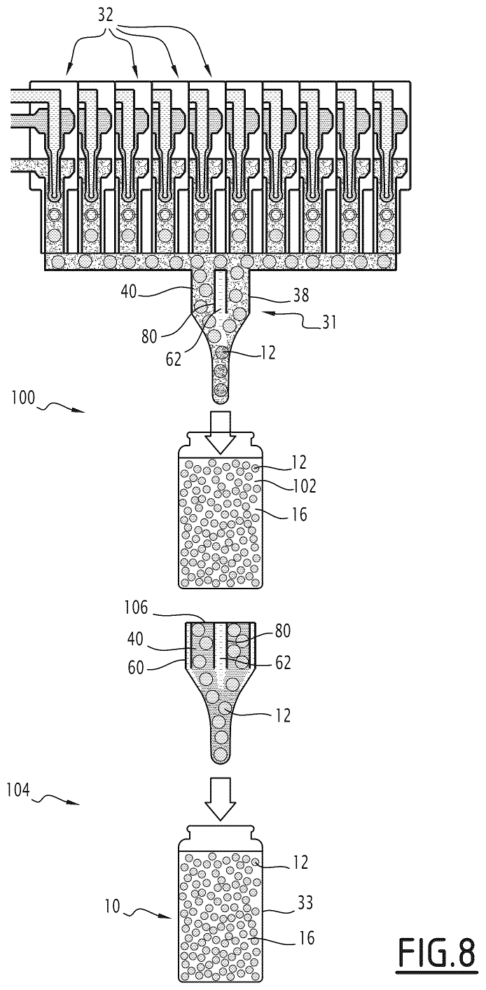

The method according to the invention can comprise one or a plurality of the following features, taken individually or according to any technically possible combination:
the drops of fatty phase and the aqueous phase flow along a local axis in the circulation duct, the injection of the additional aqueous solution (62) taking place substantially parallel, or even coaxially, with the local axis;
the injection of the additional aqueous solution (62) includes bringing at least part of the solution to the center of the flow of the drops and of the aqueous phase;
injection of the additional aqueous solution (62) includes bringing at least part of the solution to the periphery of the flow of the drops and of the aqueous phase;
the method includes a reduction in the cross-section of the flow of the drops and of the aqueous phase, downstream of the injection of the aqueous solution (62);
the injection of the aqueous solution (62) is carried out at the outlet of the circulation duct (38);
the method includes, upstream of step (iv) of flow (or of circulation), a step of formation of drops (12) in the circulation duct (38);
the injection of the aqueous solution (62) includes bringing at least part of the solution (62) into a solution dispensing ring (210) located in the center of the circulation duct (38), the drops (12) flowing through a central passage (212) delimited by the ring (210) and through a peripheral passage (214) delimited between the ring and the circulation duct (38).
according to step (vi2), a first sub-step 100 of injection of a first aqueous solution (62), suitable for increasing the viscosity of the aqueous phase (16), whereby an intermediate dispersion 102 is obtained comprising a continuous aqueous phase 22 of partially increased viscosity,
the method further comprising, after the recovery step (v), at least a second sub-step 104 comprising:
the recirculation of the intermediate dispersion 102; and
the injection of a second aqueous solution (62) into the intermediate dispersion 102.
the recirculation step includes the circulation the intermediate dispersion 102 in an additional line 106, then the recovery of the final dispersion 10 according to the invention in a container at the outlet of the additional line 106, the injection of the second aqueous solution (62) being carried out in the additional line 106, or at the outlet of the additional line 106, upstream of the container 33. Such an embodiment is in particular illustrated in FIG. 8.

A further subject matter of the invention is an apparatus for forming a dispersion 10 comprising drops 12, comprising:
a circulation duct (38) containing drops (12) of fatty phase (14) in an aqueous phase (16) substantially immiscible with the fatty phase (14);
a container (33) for the recovery of a dispersion (10) comprising drops (12) and the aqueous phase (16);
characterized in that the apparatus (30) includes:
a tank (68) containing an aqueous solution (62) comprising at least one salt;
at least one injection line (60) for injecting the aqueous solution (62), coupled to the tank (68), and coming into the circulation duct (38) or at the outlet of the circulation duct (38), upstream of the container (33),
   the fatty phase (14) comprising at least one oil and optionally at least one lipophilic gelling agent, preferably heat-sensitive; and
   the aqueous phase (16), substantially immiscible with the fatty phase (14), comprising at least water and at least one hydrophilic gelling agent apt to gel in the presence of at least one salt.

Given the foregoing, the aqueous phase (16) is advantageously free of carbomer and, optionally, the fatty phase (14) is advantageously free of amodimethicone.

The apparatus according to the invention can comprise one or a plurality of the following features, taken individually or according to any technically possible combination:
the circulation duct extends along a local axis of circulation of the drops and of the second phase, the injection line coming out coaxially with the local axis;
the apparatus includes a peripheral injection line for injecting at least part of the additional solution, coming out at the periphery of the circulation duct and/or a central injection line for injecting at least part of the additional solution, coming out at the center of the circulation duct;
the apparatus includes a unit for forming drops in the circulation duct, the unit for forming drops advantageously including:
a line for supplying a first fluid (36) comprising the fatty phase (14) and optionally at least one gelling agent, preferably thermosensitive, contained in the fatty phase (14);
a line for forming drops of first fluid (36) in a second fluid (40) intended to form the aqueous phase (16), the second fluid (40) comprising at least water and at least one hydrophilic gelling agent apt to gel in the presence of at least one salt.

A further subject matter of the invention relates to a dispersion comprising drops (12) of fatty phase (14) dispersed in a gelled continuous aqueous phase (22), wherein:
the drops (12) having a diameter greater than or equal to 100 μm represent a volume greater than or equal to 60%, or even greater than or equal to 70%, preferably greater than or equal to 80%, and better greater than or equal to 90% of the total volume of the dispersed phase and/or at least 60%, even at least 70%, preferably at least 80% and better at least 90%, of the drops have a mean diameter greater than or equal to 100 μm.
the fatty phase comprises at least one oil and optionally at least one lipophilic gelling agent, preferably thermosensitive; and
the gelled continuous aqueous phase comprises at least water, at least one hydrophilic gelling agent gelled by at least one salt,
the dispersion being free of carbomer and, optionally, of amodimethicone.

A dispersion according to the invention has the advantage of being stable (or “kinetically stable”), in particular over time and during transport. “Stable”, as defined by the present invention, refers in particular, to the absence of creaming or sedimentation of the drops of phase dispersed in the continuous phase, the absence of opacification of the continuous phase, the absence of aggregation of the drops with one another, and in particular the absence of coalescence or of Ostwald ripening of the drops between each other, and the absence of leakage of materials from the dispersed phase toward the continuous phase, or vice versa.

Within the context of the present invention, the aforementioned dispersions can be designated indifferently by the term “dispersions”.

According to another embodiment, the dispersions according to the invention do not comprise any surfactant.

Preferably, a dispersion according to the invention has a pH comprised between 3.0 and 6.5, preferably between 4.0 and 6.0, and better between 5.0 and 6.0.

Unless otherwise indicated, hereinafter, the temperature is considered to be the ambient temperature (e.g. T=25° C.±2° C.) and the pressure is considered to be the atmospheric pressure (760 mm Hg, or 1,013×10⁵ Pa or 10¹³ mbar).

The invention will be better understood upon reading the following description, given only as an example and making reference to the enclosed drawings, wherein:

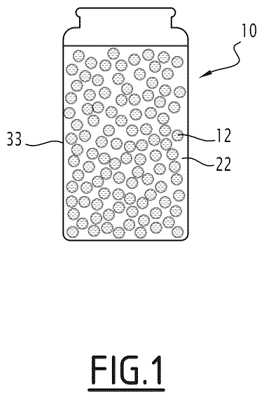

FIG. 1 is a side view of a container containing a single dispersion obtained by a second method according to the invention;

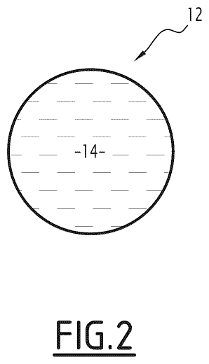

FIG. 2 is a sectional view of a drop of a single dispersion according to the invention;

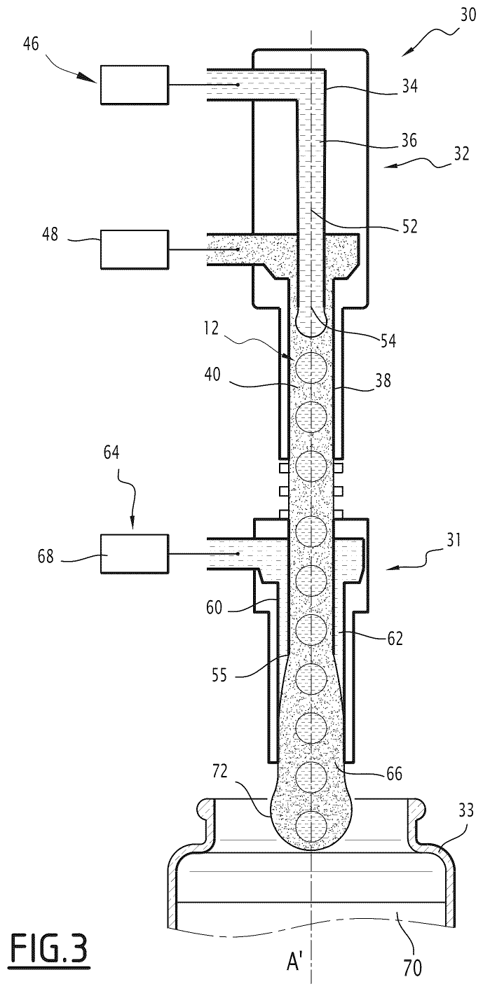

FIG. 3 is a schematic partial sectional view of a first apparatus for the manufacturing of a single dispersion, for the implementation of the second method;

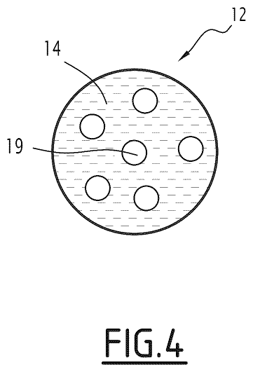

FIG. 4 is a sectional view of a drop of a multiple dispersion according to the invention;

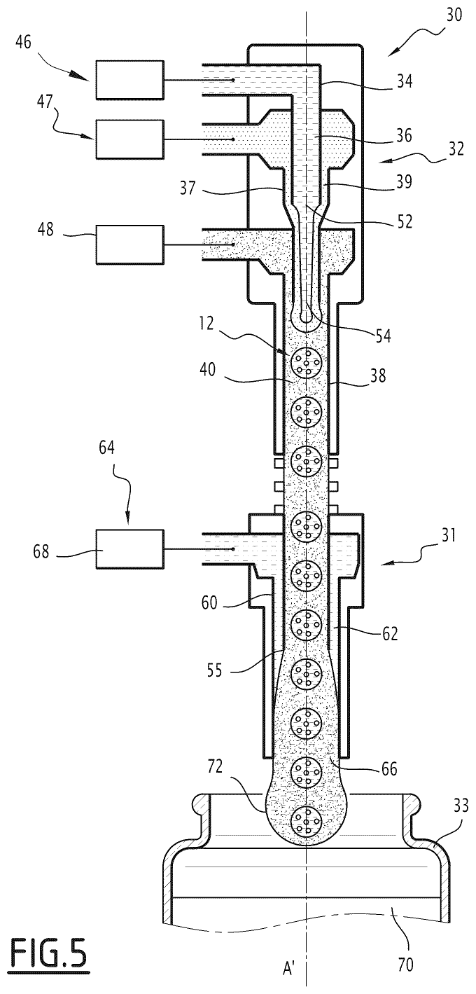

FIG. 5 is a schematic partial sectional view of a first apparatus for manufacturing a multiple dispersion, for the implementation of the second method;

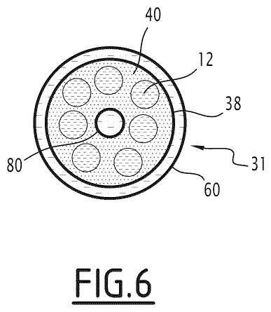

FIG. 6 is a view of a detail of the end of a circulation duct of a variant of the apparatus according to the invention, where the injection of an additional solution (62) takes place at the end of the circulation duct;

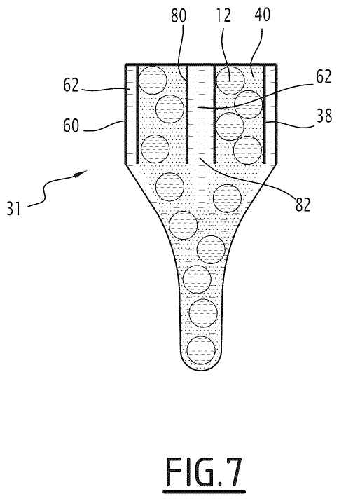

FIG. 7 is a top view of the end represented in FIG. 6; and

FIG. 8 is a view similar to FIG. 5 of a variant of an apparatus for manufacturing a multiple dispersion, for the implementation of a second method according to the invention.

FIGS. 1 to 3 illustrate the implementation of a second method for forming single dispersions according to the invention.

FIGS. 4 and 5 illustrate the implementation of a second method for forming multiple dispersions according to the invention.

### Immiscibility

The fatty phase 14 (or dispersed phase) and the aqueous phase 16 (or continuous phase) are substantially immiscible.

“Substantially immiscible”, as defined by the present invention, means that the solubility of a first phase in a second phase is advantageously less than 5% by weight.

According to a first variant embodiment, a dispersion according to the invention may be a single emulsion, and more particularly an oil-in-water direct emulsion.

According to a second variant of embodiment, a dispersion according to the invention can be a multiple emulsion and thus resort to the use of a third phase 19 (or internal dispersed phase), in which case the fatty phase 14 (or intermediate dispersed phase) is located between the third phase 19 and the aqueous phase 16 (or continuous phase), as illustrated in FIG. 4. Thereby, the fatty phase 14 and the third phase 19 are substantially immiscible and the fatty phase 14 and the aqueous phase 16 are substantially immiscible. More particularly, a multiple dispersion according to the invention is such as:
a water-in-oil-in-water dispersion, or
an oil-in-oil-in-water dispersion, in which case the third oily phase 19 and the oily fatty phase 14 comprise substantially immiscible oils.

“Substantially immiscible oils” or “non-miscible oils”, as defined by the present invention, means that the mixture of the two oils does not lead to a homogeneous single-phase solution. A person skilled in the art would be able to adjust the choice of oils to satisfy the abovementioned “immiscible” criterion. Oils immiscible with one another are described in particular in FR1752204.

According to a variant, a method according to the invention is intended to form a single final dispersion based on a transient formation step of a multiple dispersion. Thereby, in a transient multiple emulsion, the third phase 19 is oily and is miscible with the fatty phase 14.

### Continuous Aqueous Phase

A dispersion according to the invention comprises a continuous aqueous phase.

The aqueous phase according to the invention comprises water. In addition to distilled or deionized water, a water suitable for the invention can also be a natural spring water or a floral water.

According to one embodiment, the mass percentage of water of the aqueous phase is at least 30%, preferably at least 40%, more particularly at least 50%, and better at least 60%, in particular comprised between 70% and 98%, and preferentially comprised between 75% and 95%, relative to the total weight of said continuous aqueous phase.

Preferably, the continuous aqueous phase of the dispersion according to the invention does not comprise any base, more particularly NaOH.

Preferably, the continuous aqueous phase of the dispersion according to the invention does not comprise any carbomer (or acrylic polymer).

### Hydrophilic Gelling Agent Apt to Gel in the Presence of at Least One Salt

“Hydrophilic” means a gelling agent soluble or dispersible in water.

A hydrophilic gelling agent apt to gel in the presence of at least one salt, also referred to equally as “ion sensitive hydrophilic gelling agent”, is an agent which makes it possible in particular to modulate the fluidity of the aqueous phase comprising same in the presence of at least one salt, and hence the texture and/or the feeling of the dispersion. When the composition further comprises a dispersed fatty phase, a hydrophilic gelling agent apt to gel in the presence of at least one salt is an agent which also makes it possible to suspend said drops in the continuous aqueous phase.

A hydrophilic gelling agent apt to gel in the presence of at least one salt also makes it possible to provide, in full or in part, the suspensive character of the continuous aqueous phase with respect to the drops of fatty phase, and thus to provide, in full or in part, the kinetic stability of the dispersion, in particular by preventing/limiting in full or in part the phenomena of coalescence of the drops with one another and/or creaming and/or sedimentation of the drops in the continuous aqueous phase.

For obvious reasons, the choice of hydrophilic gelling agent(s) apt to gel in the presence of at least one salt should be adapted to the composition of the additional solution comprising at least one salt.

Such adaptation falls within the general skills of a person skilled in the art.

Preferably, the hydrophilic gelling agent apt to gel in the presence of at least one salt is a polyelectrolyte reactive to at least one salt, more particularly in the presence of at least one monovalent or divalent ion such as e.g., K⁺, Na⁺, Ca⁺⁺ or Mg⁺⁺.

A hydrophilic gelling agent apt to gel in the presence of at least one salt may be chosen from natural polymers, biosynthetic polymers, modified polymers, and mixtures thereof, and preferably from natural polymers.

Preferably, the hydrophilic gelling agent apt to gel in the presence of at least one salt is chosen from a polysaccharide based on algins, alginates, pectins, carrageenans, gellan gum, diutan gum, furcellaran, or one of the derivatives thereof, and mixtures thereof, more particularly from gellan gum and/or carrageenans, and most particularly from gellan gum and/or iota-carrageenans.

Preferably, the hydrophilic gelling agent apt to gel in the presence of at least one salt may be chosen from carrageenan, more particularly kappa and iota-carrageenan; gellan gum, in particular low acyl gellan; alginate; pectin, more particularly low methoxyl pectin; diutan gum; furcellaran; or one of their derivatives; and mixtures thereof.

As carrageenan, mention may be made of the reference marketed by Cargill Beauty under the name Satiagel VPC 508 P (INCI: Iota-Carrageenan (and) Chondrus Crispus Extract).

As gellan gum, mention may be made of the reference marketed by CP Kelco under the name Kelcogel CG LA or Kelcogel CG LA [E] (INCI: Gellan gum).

As alginate, mention may be made of the reference marketed by Algaia under the name Algogel VCG 1561 or by Alchemy ingredients under the name Sclerothix (INCI: Xanthan Gum (and) Sclerotium Gum (and) Algin).

Advantageously, the hydrophilic gelling agent apt to gel in the presence of at least one salt is not chosen from algin or alginate.

Preferably, the hydrophilic gelling agent apt to gel in the presence of at least one salt is not a thermosensitive hydrophilic gelling agent.

“Thermosensitive hydrophilic gelling agent” refers to a gelling agent which reacts with heat, and in particular is a gelling agent which is solid at ambient temperature and liquid at a temperature greater than 40° C., preferably greater than 50° C.

A dispersion according to the invention may comprise between 0.01% and 5%, preferably between 0.05% and 2.5%, better between 0.05% and 1%, and most particularly between 0.08% and 0.5%, by weight of hydrophilic gelling agent(s) to be gelled in the presence of at least one salt relative to the total weight of the aqueous phase (16).

Preferably, the hydrophilic gelling agents present in a gelled aqueous phase according to the invention are of natural origin and preferably biodegradable.

Preferably, the gelled aqueous phase (22) of a dispersion, or even the dispersion, according to the invention is natural, and preferably biodegradable.

“Natural”, as defined by the present invention, refers to a composition comprising a percentage of ingredients of natural origin greater than or equal to 95%, preferably greater than or equal to 96%, more particularly greater than or equal to 97%, and better greater than or equal to 98% as per the standard ISO standard 16 128. A method of calculation of percentage is described in FR3119317.

Given the manufacturing method according to the invention:
the continuous aqueous phase (16) is in liquid form at ambient temperature and atmospheric pressure. In other words, the continuous phase (16) is not solid at ambient temperature and at ambient pressure;
the gelled continuous aqueous phase (22) of the dispersion obtained after mixing between the continuous aqueous phase (16) and the aqueous solution (62) has a viscosity necessarily higher than the viscosity of the continuous aqueous phase (16) and is preferably in the form of a gel.

Advantageously, the gelled continuous aqueous phase (22) is provided with a flow threshold value suitable for providing the suspension (or ability of suspension) of the drops over a period of time greater than or equal to 1 month, preferably greater than or equal to 3 months, better greater than or equal to 6 months, and most particularly greater than or equal to 12 months. In addition to the associated visual aspect, the suspensive character serves to improve the kinetic stability of the dispersion, in particular to prevent/limit the phenomena of coalescence of the drops with one another and/or creaming and/or sedimentation of the drops in the continuous phase, and hence to prevent, furthermore, any alteration of the visual appearance of a dispersion according to the invention.

More particularly, the aqueous phase (16), and thus before mixing with the solution (62), has a viscosity as measured at 25° C., comprised between 1 mPa·s and 10,000 mPa·s, preferably from 10 mPa·s to 8,000 mPa·s, in particular from 100 mPa·s to 5,000 mPa·s, and more particularly from 200 mPa·s to 2,500 mPa·s.

More particularly, the gelled continuous aqueous phase (22) of a dispersion according to the invention, and thus after mixing the aqueous phase (16) with the aqueous solution (62), has a viscosity, as measured at 25° C., comprised between 1,000 MPa·s and 50,000 MPa·s, preferably between 2,000 MPa·s and 40,000 MPa·s, in particular between 3,000 MPa·s and 30,000 MPa·s, more particularly between 3,500 MPa·s and 20,000 MPa·s, and most particularly between 4,000 MPa·s and 10,000 MPa·s.

The viscosity is measured at ambient temperature and at ambient pressure, by the method described in WO2017046305.

The aqueous phase (22) is a non-Newtonian fluid. Same is a rheofluidifying fluid.

Preferably, the gelled aqueous phase (22) of a dispersion according to the invention is transparent or at least translucent. The transparency or translucency property is determined according to the protocol described in the examples hereafter.

The gelled aqueous phase (22) of a dispersion according to the invention is also advantageous in that same has unexpected properties in terms of shear resilience, also referred to as “viscosity regeneration time” or “reconstruction time”, i.e. the time necessary for the sample to regain a viscosity at least equal to 75% of the original viscosity.

### Method of Measuring the Regeneration Percentage:

All measurements are made with a DHR10 rheometer from Ta Instrument equipped with a 40 mm diameter mobile forming a 1° cone—the air-gap being 29 μm. The measurements are carried out at 18° C., the temperature being controlled by a Peltier device.

The thixotropic behavior is measured using a 3ITT (3-interval-thixotropy-test) test protocol. The protocol consists in successively subjecting the sample to 3 different shearing steps, namely:
First step (=reference interval): the sample is subjected to a low shear of 0.1 s⁻¹ for 30 seconds with a measurement of one second per point, by which the viscosity thereof at rest is evaluated; the measurement will serve as a reference for the test.
Second step (=high shear interval): the sample is subjected to a high shear of 300 s⁻¹ for 30 seconds, which will break the structure of the sample, with a measurement of one second per point.
Third step (=regeneration interval): the sample is subjected to the original shear, namely of 0.1 s⁻¹ for 300 seconds with a measurement of one second per point.

The time needed for the sample to return to a viscosity of at least 75% of the original viscosity is called “reconstruction time”.

Thereby, an aqueous phase (22) according to the invention advantageously has a percentage of regeneration of the viscosity thereof of at least 75%, preferably of at least 80%, more particularly of at least 85%, or even of at least 90%, after 30 seconds in the third regeneration interval according to the method described hereinabove.

An aqueous phase (22) according to the invention can thus be qualified as resilient to shear.

More particularly, the gelled continuous aqueous phase (22) of a dispersion according to the invention has a flow threshold greater than 0.1 Pa, more particularly greater than 1 Pa, and preferably comprised between 0.5 Pa and 100 Pa, more particularly between 1 Pa and 75 Pa, most particularly between 2 Pa and 50 Pa, or even between 5 Pa and 25 Pa, and better between 10 Pa and 20 Pa.

The flow threshold is measured at ambient temperature and ambient pressure using the flow shear rate sweep method described in H. A. Barnes, A Handbook of Elementary Rheology; Institute of Non-Newtonian Fluid Mechanics. University of Wales, 2000 or http://www.tainstruments.com/pdf/literature/RH025.pdf.

In addition, the continuous gelled aqueous phase (22) of a dispersion according to the invention is endowed with a satisfactory feeling, in particular of the “breakage-in-water” type, i.e. the sensation felt when an aqueous gel breaks under the pressure applied and releases the water same contains and gives a sensation of freshness and hydration.

Such feeling is unexpected because is generally not or only slightly attainable with hydrophilic gelling agents apt to gel in the presence of at least one salt according to the invention.

Such feeling is all the more unexpected as is accompanied by satisfactory properties in terms of play-time and non-stickiness.

Thereby, a method according to the invention makes it possible to obtain dispersions having an average play-time of less than 3 minutes, preferably less than 2 minutes and more preferably less than 1 minutes, when same is applied to a keratin material.

### Dispersed Fatty Phase

A dispersion according to the invention may comprise from 1% to 60%, more particularly from 5% to 50%, preferably from 10% to 40% and better from 15% to 30%, by weight of fatty phase (14) relative to the total weight of the dispersion (10).

Preferably, the fatty phase of the dispersion according to the invention does not comprise any lipophilic cationic polymer, more particularly amodimethicone (or aminosilicone).

A fatty phase (or oily phase) according to the invention comprises at least one oil, and optionally at least one lipophilic gelling agent.

### Oils

“Oil” refers to a fat that is liquid at ambient temperature.

As oils which can be used in a dispersion according to the invention, mention may be made e.g. of:
hydrocarbon oils of vegetable origin, such as hydrogenated jojoba oil, hydrogenated sunflower oil, hydrogenated castor oil, hydrogenated coconut oil;
hydrocarbon oils of animal origin, such as perhydrosqualene and squalane;
synthetic esters and ethers, in particular of fatty acids, such as oils with the formulae R1COOR2 and R1OR2 wherein R1 represents the residue of a C₈ to C₂₉ fatty acid and R2 represents a C₃ to C₃₀ hydrocarbon chain, branched or unbranched, such as Purcellin oil, isononyl isononanoate, isodecyl neopentanoate, isostearyl neopentanoate, isopropyl myristate, ethyl-2-hexyl palmitate, octyl-2-dodecyl stearate, octyl-2-dodecyl erucate, isostearyl isostearate; hydroxy esters such as isostearyl lactate, octylhydroxystearate, octyldodecyl hydroxystearate, diisostearyl malate, triisocetyl citrate, heptanoates, octanoates, decanoates of fatty alcohols; polyol esters such as propylene glycol dioctanoate, neopentyl glycol diheptanoate and diethylene glycol diisonanoate; and pentaerythritol esters such as pentaerythrityl tetrabehenate (DUB PTB) or pentaerythrityl tetraisostearate (Prisorine 3631);
linear or branched hydrocarbons of mineral or synthetic origin, such as paraffin oils, either volatile or not volatile, and the derivatives thereof, petroleum jelly, polydecenes, hydrogenated polyisobutene such as Parleam oil;
silicone oils, such as volatile or non-volatile polymethylsiloxanes (PDMS) with a linear or cyclic silicone chain, liquid or pasty at room temperature, in particular cyclopolydimethylsiloxanes (cyclomethicones) such as cyclohexasiloxane and cyclopentasiloxane; polydimethylsiloxanes (or dimethicones) containing alkyl, alkoxy or phenyl groups, during or at the end of the silicone chain, groups containing from 2 to 24 carbon atoms; phenylsilicones such as phenyltrimethicones, phenyldimethicones, phenyltrimethylsiloxydiphenylsiloxanes, diphenyldimethicones and diphenylmethyldiphenyltrisiloxanes, 2-phenylethyltrimethyl siloxysilicates, and polymethylphenylsiloxanes;
fatty alcohols with 8 to 26 carbon atoms, such as cetyl alcohol, stearyl alcohol and the mixture (cetyl stearyl alcohol) thereof, or else octyldodecanol;
partially hydrocarbon-based and/or silicone-based fluorinated oils such as same described in document JP-A-2-295912;
and mixtures thereof.

In a preferred embodiment, the oil is selected from the group consisting of isononyl isononanoate, dimethicone, isohexadecane, polydimethylsiloxane, octyldodecanol, isodecyl neopentanoate and mixtures thereof.

According to another preferred embodiment, the fatty phase does not comprise any silicone oil and preferably does not comprise any polydimethylsiloxane (PDMS).

A person skilled in the art would be able to adjust the nature and/or the content of oil(s), in particular to provide satisfactory kinetic stability of the dispersion according to the invention and to preserve the aforementioned advantageous technical effects.

A dispersion according to the invention may comprise between 30% and 100%, more particularly between 40% and 90%, preferably between 50% and 80%, and more particularly between 60% and 70%, by weight of oil(s) relative to the total weight of the fatty phase.

### Lipophilic Gelling Agent

A lipophilic gelling agent, i.e. one soluble or dispersible in the fatty phase, may be chosen from organic or inorganic, polymeric or molecular gelling agents; fats which are solid at ambient temperature and pressure, in particular chosen from waxes, pasty fats and butters; and mixtures thereof, and preferably among the polymeric gelling agents.

Such lipophilic gelling agents are described in particular in WO2019002308.

Among the lipophilic gelling agents which can be used in the present invention, mention may be made of esters of dextrin and of fatty acid, such as dextrin palmitates. Among the esters of dextrin and of fatty acid(s), mention may be made e.g. of dextrin palmitates, dextrin myristates, dextrin palmitates/ethylhexanoates and mixtures thereof. Mention may be made in particular of the esters of dextrin and of fatty acid(s) marketed under the names Rheopearl® D2 (INCI name: dextrin palmitate), Rheopearl® TT2 (INCI name: dextrin palmitate ethylhexanoate), and Rheopearl® MKL2 (INCI name: dextrin myristate) by Miyoshi Europe, also dextrin palmitate marketed by The Innovation Company.

HIXCIN® R from Elementis specialties (INCI: Trihydroxystearin), OILKEMIA™ 5S polymer from Lubrizol (INCI: Caprylic/Capric Triglyceride (and) Polyurethane-79), Estogel M from PolymerExpert (INCI: CASTOR OIL/IPDI copolymer & CAPRYLIC/CAPRIC triglyceride), hydrogenated Castor Oil/Sebacic Acid Copolymer and the derivatives thereof, in particular marketed under the names Estogel Green (or Estogel G) and Estogel Green 40, respectively, by PolymerExpert, and mixtures thereof.

Advantageously, a lipophilic gelling agent is a thermosensitive gelling agent.

Advantageously, a lipophilic gelling agent, is a thixotropic gelling agent or a gelling agent apt to impart a thixotropic behavior to the fatty phase. Such a thixotropic gelling agent is chosen in particular from pyrogenic silicas treated, if appropriate, to be hydrophobic, described hereinabove.

According to the invention, a dispersion 10 according to the invention may comprise from 0.5% to 30%, preferably from 1% to 25%, more particularly from 1.5% to 20%, better from 2% to 15%, and most particularly from 5% to 12%, by weight of lipophilic gelling agent(s) relative to the total weight of the fatty phase 14.

### Drops

The dispersed fatty phase of a dispersion according to the invention is in the form of drops, preferably macroscopic drops, i.e. visible to the naked eye.

Thereafter in the present description, the drops of fatty phase may equally well be called “drop (G1)”.

Advantageously, the dispersion according to the invention is intended to form dispersions wherein the drops (G1) having a diameter greater than or equal to 100 μm represent a volume greater than or equal to 60%, or even greater than or equal to 70%, preferably greater than or equal to 80%, and better greater than or equal to 90% of the total volume of the dispersed phase and/or at least 60%, even at least 70%, preferably at least 80% and better at least 90%, of the droplets have a mean diameter greater than or equal to 100 μm. Preferably, the diameter is greater than or equal to 150 μm, better greater than or equal to 200 μm, more particularly greater than or equal to 250 μm, preferably greater than or equal to 300 μm, more particularly greater than or equal to 400 μm and better greater than or equal to 500 μm.

Preferably, the diameter of the drops (G1) is greater than or equal to 100 microns, more particularly greater than or equal to 250 microns, preferably greater than or equal to 500 microns, and in particular comprised between 250 microns and 3000 microns, preferably between 500 microns and 2000 microns, even between 750 microns and 1500 microns.

Thereby, in a dispersion according to the invention, the constituent phases thereof form a macroscopically inhomogeneous mixture.

The droplets are advantageously substantially spherical.

Advantageously, the drops advantageously have an apparent monodispersity (i.e. same are perceived by the eye as spheres identical in diameter).

Preferably, the dispersions of the invention consist of a population of monodispersed droplets, in particular such that same have a mean diameter D̅ comprised between 100 μm to 3,000 μm, more particularly from 500 μm to 3,000 μm and a coefficient of variation Cv of less than 10%, or even less than 3%.

Within the framework of the present description, “monodispersed droplets” refer to the fact that the population of droplets of the dispersion according to the invention has a uniform size distribution. Monodisperse droplets exhibit good monodispersity. On the other hand, droplets exhibiting poor monodispersity are called “polydispersed”.

According to one embodiment, the mean diameter D̅ of the droplets is measured, e.g., by analyzing a photograph of a batch consisting of N droplets, by image methoding software (Image J). Typically, according to such method, the diameter is measured in pixels, then related to μm, as a function of the size of the container containing the droplets of the dispersion.

Preferably, the value of N is chosen to be greater than or equal to 30, so that such analysis statistically significantly reflects the distribution of diameters of the droplets of said emulsion. N is advantageously greater than or equal to 100, in particular in the case where the dispersion is polydispersed.

The diameter Di of each droplet is measured, then the mean diameter is obtained D̅ by calculating the arithmetic mean of the values:

$$\overline{D} = \frac{1}{N} {\sum }_{i = 1}^{N} {D}_{i}$$

From the Di values, the standard deviation σ of the diameters of the droplets of the dispersion can be also obtained:

$$σ = \sqrt{\frac{{\sum }_{i = 1}^{N} {( {D}_{i} - \overline{D} )}^{2}}{N}}$$

The standard deviation σ of a dispersion reflects the distribution of the diameters Di of the droplets of the dispersion around the mean diameter D̅.

By knowing the mean diameter D̅ and the standard deviation of σ a dispersion, it can be determined that 95.4% of the droplet population is found in the interval of diameters [D̅−2σ;D̅+2σ] and 68.2% of the population is found in the interval [D̅−σ;D̅+σ].

To characterize the monodispersity of the dispersion according to said embodiment of invention, the coefficient of variation can be calculated:

$${C}_{v} = \frac{σ}{\overline{D}}$$

Such parameter reflects the distribution of the diameters of the droplets according to the mean diameter of the latter.

The coefficient of variation Cv of the diameters of the droplets according to said embodiment of the invention is less than 10%, preferably less than 5%, or even less than 3%.

Alternatively, the monodispersity can be demonstrated by placing a dispersion sample in a bottle with a constant circular cross-section. Gentle stirring by a quarter-turn rotation of the bottle in one half-second about the axis of symmetry passing through the bottle, followed by a rest time of one half-second is performed before repeating the operation in the opposite direction, the whole operation being repeated four successive times.

The droplets of the dispersed phase organize in a crystalline form when the droplets are monodispersed. Thereby, the droplets are stacked in a pattern repeating in three dimensions. It is then possible to observe a regular stacking which indicates a good monodispersity, an irregular stacking reflecting the polydispersity of the dispersion.

Such a monodisperse character is a direct consequence of the microfluidic method according to the invention.

Drops can be monophasic or multiphasic. For example, the drops comprise a core (which comprises at least the fatty phase), optionally a shell (or envelope or membrane) totally encapsulating the core, where the core as such can comprise one or a plurality of phases.

According to a first embodiment, a drop according to the invention is a solid (or monophasic) particle.

According to a second embodiment, a drop according to the invention is a core/shell particle. A core/shell drop is a capsule which comprises a core, preferably liquid or at least partially gelled or at least partially thixotropic, and a shell, completely encapsulating said core, said core being monophasic, and hence containing the fatty phase.

A solid pearl drop is illustrated in FIG. 2.

According to such variant, a drop may also be a solid or core/shell particle including an intermediate drop (G1) of an intermediate fatty phase, the intermediate phase (or fatty phase 14) being placed in contact with the aqueous phase 16 or with the shell (when present), and at least one, preferably a single, internal drop (G2) of an inner phase (or third phase 19) arranged in the intermediate drop (G1). Such a complex drop is illustrated in FIG. 4.

According to such variant, the intermediate fatty phase advantageously comprises at least one lipophilic gelling agent, in particular as defined hereinabove, in particular to improve the suspension of the drop(s) (G2) disposed in the intermediate drop (G1) and thereby prevent/avoid creaming or sedimentation phenomena of the drop(s) (G2).

Preferably, the dispersed fatty phase is transparent or at least translucent. The transparency or translucency property of the dispersed phase is determined as follows: the composition to be tested (30 ml) is poured into a 30 ml Volga jar, the composition is left for 24 hours at ambient temperature and a white sheet is placed beneath, on which a cross approximately 2 mm thick is drawn with a black felt. If the cross is visible to the naked eye in daylight at an observation distance of 40 cm, the composition is transparent or translucent.

According to one particular embodiment:
the continuous aqueous phase of a dispersion according to the invention may as such be in the form of a direct emulsion comprising a dispersed fatty phase in the form of drops (G3) the size of which is preferably smaller than the size of the drops (G1), or even of the drops (G2); and/or
the dispersed fatty phase, or even the intermediate fatty phase and/or the inner phase in the case of a multiple dispersion (or complex drop) as defined hereinabove, may be in the form of a direct or invert emulsion comprising drops (G4) and/or (G5), the size of the drops (G4) and/or (G5) being necessarily smaller than the size of the drops (G1), or even of the drops (G2).

The drops (G3) and/or (G4) and/or (G5) are preferably microscopic, i.e. not visible to the naked eye and in particular of a size less than 100 μm, preferably less than 20 μm and better less than 10 μm.

In other words, the drops (G3) and/or (G4) and/or (G5) are different and independent from the drops (G1), or even of the drops (G2).

The drops (G1) of a dispersion according to the invention are advantageously free of any shell, more particularly of any polymeric membrane or a membrane formed by interfacial polymerization. More particularly, the droplets (G1) of a dispersion according to the invention are not stabilized using a coacervate (such as anionic polymer (carbomer)/cationic polymer (amodimethicone)) membrane. In other words, the contact between the continuous aqueous phase and the dispersed fatty phase is preferably direct.

According to another embodiment, the drops (G1) comprise a shell.

The presence of a shell advantageously enhances the kinetic stability of the drops (G1), and hence the dispersion.

### Aqueous Solution Comprising at Least One Salt

The aqueous solution comprises at least one salt which acts as a gelling activator of the ion sensitive hydrophilic gelling agent.

Of course, the salt(s) is/are chosen from salts apt to react with the hydrophilic gelling agent apt to gel in the presence of at least one salt. In other words, the choice of salt(s) present should be adjusted with regard to the hydrophilic gelling agent apt to gel in the presence of at least one salt.

“Apt to react with the gelling agent apt to gel in the presence of at least one salt” refers to a salt apt to modulate and most particularly increase, the viscosity of an aqueous phase comprising hydrophilic gelling agents apt to gel in the presence of at least said salt.

The aqueous solution comprising at least one salt (or “additional solution” or “BF” or “solution 62”) is miscible with the continuous aqueous phase 16.

Such additional solution, added to the aqueous phase (16) before step (iii) and/or after step (iv), has the effect of interacting with the gelling agent apt to gel in the presence of at least one salt, and thereby to induce the gelling thereof and thus an increase in the suspension (i.e. of the flow threshold), or even the viscosity, of the aqueous phase 16, whereby a continuous gelled aqueous phase (22) is obtained.

As indicated hereinbelow, the viscosity of the aqueous phase 16 of the dispersion is advantageously increased in order to maintain the drops 12 in suspension, preferably over a period of time of at least 1 month, preferably at least 3 months, better at least 6 months, and most particularly at least 12 months.

An additional solution according to the invention is an aqueous solution which comprises at least water. In addition to distilled or deionized water, a water suitable for the invention can also be a natural spring water or a floral water.

According to one embodiment, the mass percentage of water of the additional solution is at least 30%, preferably at least 40%, more particularly at least 50%, and better at least 60%, in particular comprised between 70% and 98%, and preferentially comprised between 75% and 95%, relative to the total weight of said additional solution.

An additional solution according to the invention is an aqueous solution which additionally comprises at least one salt, more particularly at least one monovalent or divalent ion such as, e.g., K+, Na+, Ca++ or Mg++.

The salt may be chosen from a monovalent salt, preferably from sodium salts such as sodium chloride, potassium salts such as potassium chloride; or multivalent, in particular divalent, preferably from calcium salts such as calcium chloride, calcium gluconate, calcium citrate, calcium carbonate, magnesium salts such as magnesium sulfate, and mixtures thereof, and preferably the salt is a monovalent salt, more particularly sodium chloride.

Preferably, the additional solution does not comprise any carbomer (or acrylic polymer).

Preferably, the additional solution does not comprise any base, in particular NaOH.

Thereby, an additional solution is different from a solution for increasing the viscosity as described in WO2015055748.

Of course, a person skilled in the art would make sure to select the salt(s) and/or the amount thereof depending on the gelling agent considered, and in particular the aptitude thereof to gel in the presence of at least one salt, the solubility limit of the salt(s) considered, and also in such a way that the advantageous properties of a dispersion according to the invention are not or are not substantially altered by the envisaged addition. Such adjustments fall within the general knowledge of a person skilled in the art.

The aqueous solution (62) may advantageously comprise between 0.01% and 30%, preferably between 0.1% and 20%, better between 1% and 15%, even between 2% and 10%, by weight of salt(s) relative to the total weight of the aqueous solution (62).

The weight ratio “hydrophilic gelling agent(s) apt to gel in the presence of at least one salt/salt(s)” is advantageously comprised between 0.1 and 20, better between 0.2 and 20, more particularly between 0.5 and 15, preferably between 1 and 10, and better between 2.5 and 5.

### Additional Compound(s)

According to the invention, the continuous aqueous phase and/or the dispersed fatty phase and/or the additional solution, or even the third phase 19, may further comprise at least one additional compound different from the abovementioned oils and gelling agents.

According to the invention, the continuous aqueous phase and/or the dispersed fatty phase and/or the additional solution, or even the third phase 19, may thereby further comprise powders; coloring agents, in particular chosen from coloring agents, either hydrosoluble or not, either liposolubles ou not, organic or inorganic, optical effect materials, liquid crystals and mixtures thereof; fillers, more particularly pigments and/or nacres, such as described in FR3067930; emulsifier and/or non-emulsifier silicone elastomers, in particular as described in EP2353577; additional hydrophilic gelling agents (or texture agents) different from a hydrophilic gelling agent apt to gel in the presence of at least one salt described hereinabove; glycerin; preservatives; humidifiers; stabilizers; pH-stabilizer agents, more particularly a pH buffer (e.g. HEPES, PBS); chelating agents; softeners; retarding agents; etc. or any usual cosmetic additive; and mixtures thereof.

Also, the continuous aqueous phase and the dispersed fatty phase and/or the additional solution, or even the third phase 19 may further comprise at least one biological and/or cosmetic active agent chosen from moisturizing agents, cicatrizing agents, depigmenting agents, UV filters, desquamating agents, antioxidant agents, active agents stimulating the synthesis of dermal and/or epidermal macromolecules, skin-relaxing agents, antiperspirant agents, soothing agents, anti-aging agents, scenting agents, anticoagulants, antithrombogenic agents, anti-mitotic agents, anti-proliferation agents, anti-adhesion, anti-migration, cell adhesion promoters, growth factors, antiparasitic molecules, anti-inflammatories, angiogenesis inhibitors, vitamins, hormones, proteins, antifungals, antimicrobial molecules, antiseptics or antibiotics and mixtures thereof. Such active ingredients are described in particular in FR 1,558 849.

### Additional Hydrophilic Gelling Agent

According to a particular embodiment, the continuous aqueous phase 16 and/or the additional solution, or even the third phase 19, further comprises at least one additional hydrophilic gelling agent (or “hydrophilic texture agent”), different from a hydrophilic gelling agent apt to gel in the presence of at least one salt described hereinabove.

As additional hydrophilic gelling agents, i.e. soluble or dispersible in water, mention may be made of:
natural gelling agents, in particular chosen from extracts of algae, exudates of plants, extracts of seeds, exudates of microorganisms, such as alcasealan (INCI: Alcaligenes Polysaccharides), and other natural agents, more particularly hyaluronic acid,
semi-synthetic gelling agents, in particular chosen from cellulose derivatives and modified starches,
synthetic gelling agents, in particular chosen from homopolymers of (meth)acrylic acid or one of the esters thereof, copolymers of (meth)acrylic acid or one of the esters thereof, copolymers of AMPS (2-acrylamido-2-methylpropanesulphonic acid), associative polymers,
other gelling agents, chosen from clays, silicas such as same marketed under the names Aerosil® 90/130/150/200/300/380), and
the mixtures thereof.

“Associative polymer”, as defined by the present invention, refers to any amphiphilic polymer comprising in the structure thereof at least one fatty chain and at least one hydrophilic portion; the associative polymers according to the present invention may be anionic, cationic, nonionic or amphoteric; same described in FR 2 999 921 are concerned. Preferably, same are amphiphilic and anionic associative polymers and amphiphilic and nonionic associative polymers as described hereinafter. The additional hydrophilic gelling agents are described in more detail in FR3041251.

An additional hydrophilic gelling agent is in particular Sucraclear HC-31 (INCI: Chondrus Crispus Powder (and) cellulose Gum (and) Ceratonia Siliqua (Carob) Gum (and) glucose) or Sucraclear V2 (INCI: Cellulose Gum, Chondrus Cripsus Powder (Carageenan), Ceratonia Siliqua Gum, glucose), preBIULIN C90 (INCI: Cellulose Gum (and) Xanthan Gum (and) Inulin (and) Cellulose (and) Glucose (and) Fructose), and mixtures thereof.

The aqueous phase may further comprise at least one retarding agent. The presence of such a retarding agent in aqueous phase (16) or (22) according to the chosen manufacturing method advantageously makes it possible to reduce the kinetics of gelation of the ion sensitive hydrophilic gelling agent, and thereby to prevent clogging in the microfluidic channels and thus to mechanically improve, the stability and robustness of the manufacturing method. A retarding agent is preferably a chelating agent, more particularly chosen from at least one organophosphate, and better is tetrasodium pyrophosphate.

Of course, a person skilled in the art would make sure to choose the possible additional compound(s) of the composition and/or the amount thereof so that the advantageous properties of a dispersion according to the invention, are not altered or not substantially altered by the envisaged admixture. Also, a person skilled in the art will make sure to choose the nature and/or the amount of additional compound(s) depending on the aqueous or fatty nature of the phase considered and/or with regard to the method for manufacturing the dispersion.

Such adjustments fall within the general knowledge of a person skilled in the art.

### METHOD

### Steps (i) and (ii)

Steps (i) and (ii) of a method according to the invention fall within the general skills of a person skilled in the art.

Concerning step (ii), the addition in aqueous phase 16 of the hydrophilic gelling agent apt to gel in the presence of at least one salt can be carried out at ambient temperature or at a temperature higher than the ambient temperature, in particular at a temperature comprised between 60° C. and 90° C. Preferably, when the addition is carried out at a temperature lower than 60° C., e.g. at ambient temperature, the aqueous phase further comprises at least one sequestering agent, chosen from sodium citrate, phosphate, ethylene-diamine-tetra-acetic acid (or EDTA), Trisodium-ethylenediamine Disuccinate, e.g. marketed by Innospec under the name Natrlquest E30, sodium gluconate phytic acid, and mixtures thereof. The presence of such a sequestering agent makes it possible to reduce the hydration temperature of the hydrophilic gelling agent apt to gel in the presence of at least one salt.

### Steps (iii) and (iv)

Steps (iii) and (iv) of a method according to the invention can be carried out according to a microfluidic method such as described in WO2012/120043, WO2015/055748 or WO2019/145424.

### Step (vi)

The first method according to the invention comprises a step (vi) based at least on step (vi1) described hereinabove. Given the foregoing, step (vi1) precedes step (iii), and thus precedes the bringing into contact between the aqueous phase 16 and the fatty phase 14.

More particularly, step (vi1) is considered when a method according to the invention comprising a step (vi2) leads to dispersions wherein the viscosity of the continuous gelled aqueous phase (22) is high, which is undesirable. Indeed, under certain conditions, the inventors observed, in a dispersion obtained with a method according to the invention comprising a step (vi2), that the viscosity of the gelled continuous aqueous phase (22) is sometimes devolutive.

However, with such a method according to the invention based on step (vi2), lowering the content of hydrophilic gelling agent apt to gel in the presence of at least one salt and/or the salt content can certainly lead to lowering the viscosity of the gelled continuous aqueous phase (22) but sometimes to the detriment of the suspension with respect to the drops (G1), which is undesirable.

Against all expectations, the inventors observed that it was possible to initiate the formation of, or even to form, the gelled continuous aqueous phase (22) before step (iii), while remaining compatible with the microfluidic constraints.

More particularly, step (vi1) comprises, prior to step (iii), at least the following steps:
(a) adding the aqueous solution (62) to the aqueous phase (16), preferably at a temperature above the ambient temperature, more particularly at a temperature comprised between 40° C. and 100° C., or even between 50° C. and 90° C., and most particularly between 60° C. and 80° C.;
(b) returning the mixture obtained in step (a) to ambient temperature; and
(c) optionally, shearing the mixture obtained in step (b).

Step (a) is advantageously carried out at a temperature above the ambient temperature in order to improve, or even accelerate, the production of a homogeneous mixture between the aqueous solution (62) and the aqueous phase (16).

Step (b) ensures a return to the ambient temperature of the mixture obtained in step (a), whereby the viscosity of said mixture increases with time until same reaches the maximum value thereof.

According to a first variant, step (b) is carried out at ambient temperature for a sufficient time for the viscosity of the mixture obtained in step (a) to reach the maximum value thereof, where the time may be comprised between 30 minutes and 5 hours, preferably between 1 hour and 3 hours.

Said time falls within the general skills of a person skilled in the art.

According to a second variant, step (b) is carried out at a temperature less than the ambient temperature, more particularly at a temperature less than or equal to 20° C., preferably less than or equal to 15° C., or even less than or equal to 10° C. The second variant is advantageous in that same makes it possible to accelerate the cooling of the gelled continuous aqueous phase (22) and hence to obtain more rapidly the maximum viscosity of the gelled continuous aqueous phase (22).

Step (c), optional, is intended to shear the mixture obtained during step (b). The shearing serves to reduce the viscosity of the gelled continuous aqueous phase (22) without altering the suspensive properties thereof and without altering the visual appearance thereof, in particular the transparency. The shearing also makes it possible to optimize the stability of the viscosity and of the suspensive power thereof, in particular when the gelled continuous aqueous phase (22) is subsequently subjected to heating, e.g. to a temperature between 50° C. and 90° C., as well as the resistance of the gelled continuous aqueous phase (22) to shearing and oscillations.

The second method according to the invention comprises a step (vi) based at least on step (vi2) described hereinabove.

The second method according to the invention is implemented using a microfluidic method, in an apparatus 30 as illustrated in FIG. 3.

The apparatus 30 includes a nozzle 32 for forming the drops 12, a stage 31 for injecting the additional solution and a container 33 for receiving the drops 12 formed.

In the case of a single dispersion, the forming nozzle 32 includes at least one internal line 34 for bringing an internal fluid 36 comprising the fatty phase 14, and an external circulation duct 38, arranged around the internal line 34 for bringing and making flow an external fluid 40 comprising the aqueous phase 16.

The apparatus 30 further includes conveying means 46 for bringing the internal fluid 36 into the internal line 34 and conveying means 48 for bringing the external fluid 40 into the annular space delimited between the internal line 34 and the external line 38.

In the example shown in FIG. 3, the maximum diameter of the lines 34 and 38 is less than 3 mm, so as to preserve the microfluidic character of the method.

The internal line 34 is advantageously disposed coaxially in the external line 38. Same is connected upstream to the conveying means 46. Same opens downstream via a downstream opening 54 arranged in the external line 38.

The external line 38 delimits with the internal line 34, an annular space connected upstream to the conveying means 48.

The external line 38 has a downstream opening 55 which is situated above and at a distance from the container 33. Same comes out into the stage of injection of the solution 62.

The conveying means 46 and 48 each include e.g. a syringe pusher, a peristaltic pump or another pressure generating system controlling the flow-rate, such as e.g. a pressure pot coupled to a flow meter and a flow regulation system.

Each of the conveying means 46 and 48 is suitable for conveying a respective fluid 36 and 40 at a controlled and adjustable flow-rate.

In the case of a multiple dispersion, the formation nozzle 32 includes at least one internal line 34 for bringing a third internal fluid 36 comprising the third phase 19, and an intermediate line 37 for bringing an intermediate fluid 39 intended to form the fatty phase 14 arranged around the internal line 34.

Such embodiment is illustrated in FIGS. 4 and 5.

The formation nozzle 32 further includes an external circulation duct 38, arranged around the internal line 34 and/or the intermediate line 37 for bringing and making flow, an external fluid 40 comprising the aqueous phase 16.

The apparatus 30 further includes conveying means 46 for bringing the internal fluid 36 into the internal line 34, conveying means 47 for bringing intermediate fluid 39 into the intermediate line 37, and conveying means 48 for bringing the external fluid 40 into the annular space delimited between the internal line 34 and the external line 38.

In the example shown in FIG. 5, the maximum diameter of the lines 34, 37 and 38 is less than 3 mm, so as to preserve the microfluidic character of the method.

The internal line 34 is advantageously disposed coaxially in the external line 38. Same is connected upstream to the conveying means 46. Same comes out downstream via a downstream opening 52 arranged in the external line 38, set back with respect to the downstream opening 54 defined by the intermediate line 37, above the opening 54.

According to a first variant, the distance separating the downstream opening 52 of the internal line 34 and the downstream opening 54 of the intermediate line 37 is preferably greater than 1 times the diameter of the intermediate line 37.

According to a second variant, the distance separating the downstream opening 52 of the internal line 34 and the downstream opening 54 of the intermediate line 37 is preferably less than 1 times the diameter of the intermediate line 37, even advantageously, the downstream opening 52 of the internal line 34 and the downstream opening 54 of the intermediate line 37 are located on the same horizontal plane.

The intermediate line 37 extends around the internal line 34. Same delimits with the internal line 34, an annular space connected upstream to the conveying means 47. The intermediate line 37 comes out through the downstream opening 54.

The external line 38 delimits with the intermediate line 37 and/or the internal line 34 an annular space coupled upstream to the conveying means 48.

The external line 38 has a downstream opening 55 which is situated above and at a distance from the container 33. Same comes out into the stage 31 of injection of the additional solution.

The conveying means 46, 47 and 48 each include e.g. a syringe pusher, a peristaltic pump or another pressure generating system controlling the flow-rate, such as, e.g., a pressure pot coupled to a flow meter and to a flow-rate regulating system.

Each of the conveying means 46, 47 and 48 is suitable for conveying a respective fluid 36, 39, 40 at a controlled and adjustable flow-rate.

For the two variants of embodiment of the second manufacturing method described hereinabove, the stage 31 includes at least one line 60 for injecting a solution 62, and means 64 for bringing the solution 62 into the line 60.

In the example shown in FIG. 3, the stage 31 includes a peripheral line 60 for injecting the solution 62.

The peripheral line 60 extends at the periphery of the external circulation duct 38, in parallel, and in the present case coaxially, the local axis of the external line 38. The downstream opening 55 of the external line 38 extends into the peripheral line 60.

The peripheral line 60 defines, downstream of the downstream opening 55, a dispensing opening 66 which comes into the container 33 or above same.

The peripheral line 60 delimits, with the external line 38, an annular space which comes out upstream of the dispensing opening 66 in the example shown in FIG. 3.

Thereby, the peripheral line 60 is configured to allow the solution 62 to be injected coaxially with the axis of circulation of the dispersion containing drops 12 and the aqueous phase 16, just at the outlet of the external circulation duct 38.

In the present example, the peripheral line 60 is suitable for collecting the drops 12 and the aqueous phase 16 into which the solution 62 has been fed, and to convey same to the dispensing opening 66.

The conveying means 64 include a tank 68 containing the solution 62, and a conveying unit (not shown).

The conveying unit includes e.g. a syringe pusher, a peristaltic pump or another pressure generating system controlling the flow-rate, such as a pressure pot coupled with a flow meter and a to a flow regulation system.

For the two alternative embodiments of the second manufacturing method described hereinabove, the container 33 is arranged below the dispensing opening 66.

In a variant, the container contains a volume 70 of liquid intended to form part of the second phase 16, advantageously a volume of external fluid 40.

Also, the upper surface of the volume 70 of fluid is situated axially away from the dispensing opening 66, taken along the axis A-A′ of the line 60, in such a way that the drops 12 dispersed in the second phase 16 drop under the effect of the weight thereof through a volume of air between the dispensing opening 66 and the upper surface of the volume 70 of liquid. In a variant (not shown), the downstream opening 66 is immersed in the volume of liquid 70.

In the examples shown in FIGS. 3 and 5, the device 30 was illustrated with only one nozzle 32, associated with only one stage 31.

In an advantageous variant, illustrated in FIG. 8, the system 30 includes a plurality of nozzles 32, all connected downstream to a common stage 31, the nozzles 32 being arranged in parallel above a container 33. At least one of the nozzles 32 are laterally offset with respect to the stage 31. A collection circuit serves to collect the drops 12 in the liquid 40 at the outlet of each nozzle 32 in order to collect same and feed same into the stage 31.

A second method according to the invention intended for manufacturing a single dispersion, implemented in the installation shown in FIG. 3, will now be described.

Initially, the internal fluid 36 is prepared. The fatty phase 14 contains at least one oil, and optionally at least one lipophilic gelling agent, more particularly a thermosensitive agent.

The external fluid 40 is also prepared. The aqueous phase 16 contains at least water and at least one hydrophilic gelling agent apt to gel in the presence of at least one salt.

For obvious reasons, the steps of mixing the constituent compounds of the fatty phase 14 and of the aqueous phase 16 are carried out under conditions suitable for forming fluid phases compatible with the microfluidic method according to the invention. More particularly, and if need be, the step of mixing the constituent compounds of the fatty phase and/or the step of mixing the constituent compounds of the fatty phase takes place under hot conditions, and in particular at a temperature comprised between 6° and 100° C., preferably between 7° and 90° C. Such is in particular the case when a phase comprises at least one thermosensitive gelling agent or for facilitating the incorporation of a raw material into the solvent considered.

Advantageously, the internal fluid 36 and/or the external fluid 40 may also comprise at least one additional compound as defined hereinabove.

An aqueous solution 62 is also prepared.

The internal fluid 36 and the external fluid 40, respectively, are then arranged in the respective conveying means 46 and 48.

The aqueous solution 62 is arranged in the conveying means 64.

Optionally, a liquid 70, formed of an aqueous solvent of a nature similar to the nature of the external fluid 40, is fed into the container 33.

The conveying means 46, 48 and 64 are then activated.

The flow of the internal fluid 36 circulating in the internal line 34 enters coaxially into the line 38 at the downstream opening 54 of the internal line 34.

At the downstream opening 54 of the internal line 34, drops 12 of internal fluid 36, are surrounded by an external fluid film 40.

The drops 12 then circulate in the external fluid 40 toward the downstream opening 55.

The drops 12 in the external fluid 40 then arrive into the stage 31.

The solution 62 is then injected coaxially with the flow of drops 12 into the external fluid 40, at the periphery of the external fluid 40. The solution 62 diffuses into the external fluid 40 during the transport thereof through the downstream part of the peripheral line 60.

Thereby, the viscosity of the external fluid 40 is increased, after formation of the drops 12, in particular to ensure a satisfactory suspension of the drops 12 in the external fluid 40 and/or to achieve the desired texture.

All or a part of the increase in viscosity takes place in the vicinity of the dispensing opening 66, before, simultaneously and/or after the injection of the solution 62 before feeding the dispersion 10 into the container 33.

At least one drop 12 is then received in an external drop 72 of external fluid 40 which is formed at the outlet of the peripheral line 60, at the dispensing opening 66.

The outer drop 72 falls into the container 33, where appropriate through a volume of air and the drops 12 of the first phase 14 remain suspended in the gelled aqueous phase 22 formed by the external fluid 40, the aqueous solution 62, or even by the liquid 70 when such a liquid is present in the container 33.

In a variant, the gelled aqueous phase 22 forms a jet at the outlet of the peripheral line 60 and is collected without being fragmented.

The injection of the solution 62 and the increase in viscosity of the aqueous phase 16, are thus carried out in quite non-invasive way, and directly in line with the manufacture of the drops 12.

The above guarantees the use of an aqueous phase that is sufficiently fluid to permit an adequate formation of drops 12 at the nozzle 32 while guaranteeing a robust method of manufacturing the drops. Nevertheless, the final product comprises a continuous phase with a satisfactory viscosity to impart to same a pleasant texture and a satisfactory suspension of the drops 12 formed, via a continuous, simple, secure and low cost manufacturing method.

The method according to the invention is thus particularly effective for forming stable drops 12, of dimensions greater than 100 μm, more particularly greater than 250 μm, better greater than 500 μm, in stable suspension in a gelled aqueous phase 22, without the use of any carbomer or surfactant and in a particularly controlled manner.

The method according to the invention limits the shear, since the continuous aqueous phase 16 containing the drops 12 remains fluid until the last moment. No force is generated to deform or fragment the drops 12 when the solution 62 is injected.

Creaming is also reduced. The diffusion time of the solution 62 in the continuous phase 16 is very short, given the small thickness to get through. The continuous phase 16 almost immediately acquires a suspensive character when same is collected in the container 33.

In the variant shown in FIGS. 6 and 7, the peripheral line 60 comes out just at the outlet of the external line 38. The downstream edge of the peripheral line 60 is situated at the same horizontal level as the downstream edge of the external line 38.

The dispensing opening 66 is then situated at the same horizontal level as the downstream opening 55 of the external circulation duct 38.

Furthermore, the stage 31 comprises a central line 80 for the injection of at least part of the solution 62, which extends to the center of the external circulation duct 38.

In the present example, the central line 80 comes out just at the outlet of the external line 38. The downstream edge thereof is situated at the same horizontal level as the downstream edge of the external line 38.

The dispensing opening 82 of the central line 80 is thus situated at the same horizontal level as the downstream opening 55 of the external circulation duct 38 and the dispensing opening 66 of the peripheral line 60.

Such conformation reduces the thickness of the flow comprising the drops 12, the external fluid 40, and the solution 62, since the flow becomes thinner by gravity by penetrating into the volume of air situated at the outlet of the lines 38, 60, 80, as illustrated in FIG. 7.

The mixing of the solution 62, or even the increase in viscosity, in the external fluid 40 is then very homogeneous.

A person skilled in the art will make sure to adjust the parameters of the microfluidic manufacturing method to guarantee the proper functioning thereof, in particular so as to ensure the use of phases with an appropriate fluidity which can be reached in particular by a rise in the temperature of said phases and/or a sufficient extemporaneous shear.

### Uses

Preferably, a dispersion according to the invention can be used directly, at the end of the aforementioned preparation methods, as a composition, in particular a cosmetic composition.

The invention further relates to the use of a dispersion according to the invention for the preparation of a composition, in particular a cosmetic, pharmaceutical, nutritional or food-processing composition, preferably a cosmetic composition and more particularly a care composition and/or a make-up composition for a keratin material, more particularly the skin.

The present invention thereby further relates to a composition, in particular a cosmetic composition, more particularly a care composition and/or a make-up composition for a keratin material, in particular the skin and/or the hair, and more particularly the skin, comprising at least one dispersion according to the invention, optionally in combination with at least one physiologically acceptable medium.

The dispersions or compositions according to the invention can thus be used in particular in the cosmetic field.

Same may comprise, in addition to the abovementioned ingredients or compounds, at least one physiologically acceptable medium.

The physiologically acceptable medium is generally suitable for the nature of the support to which the composition is to be applied, as well as to the appearance wherein the composition is to be packaged.

According to one embodiment, the physiologically acceptable medium is represented directly by the aqueous continuous phase as described hereinabove.

Within the framework of the invention, and unless otherwise mentioned, a “physiologically acceptable medium” means a medium which is suitable for cosmetic applications, and which is suitable in particular for the application of a composition of the invention to a keratin material, in particular the skin and/or the hair, and more particularly the skin.

The cosmetic compositions of the invention may be e.g. a cream, a lotion, a serum and a gel for the skin (hands, face, feet, etc.), a foundation (liquid, paste) or a preparation for bath and shower (salts, foams, oils, gels, etc.), a hair care product (hair dyes and bleaches), a cleansing product (lotions, powders, shampoos), a hair care product (lotions, creams, oils), a styling product (lotions, lacquers, brilliants), a shaving product (soaps, foams, lotions, etc.), a product intended to be applied to the lips, a suncare product, a tanning product in the absence of sun, a skin whitening product, and an antiwrinkle product. More particularly, the cosmetic compositions of the invention may be an anti-aging serum, a youth serum, a moisturizing serum or a fragrant water.

According to one embodiment, the compositions of the invention are in the form of a foundation, a make-up remover, a facial and/or body and/or hair care, an anti-aging care, a sun protector, a greasy skin care, a whitening care, a moisturizing care, a BB cream, a tinted cream or foundation, a face and/or body cleanser, a shower gel or shampoo.

Thereby, given the foregoing, a dispersion or composition according to the invention is oral or topical, preferably topical, and better topical on a keratin material, more particularly the skin, and better the face skin.

A care composition according to the invention may in particular be a sunscreen composition, a care cream, a serum or a deodorant.

The compositions according to the invention may be in various forms, in particular in the form of cream, balm, lotion, serum, gel, gel-cream or else mist.

Thereby, given the foregoing, a composition according to the invention is oral or topical, preferably topical, and better topical on a keratin material, more particularly the skin, and better the face skin.

The present invention further relates to a non-therapeutic method for the cosmetic treatment of a keratin material, in particular the skin and/or the hair, and more particularly the skin, comprising a step of applying to said keratin material at least one dispersion or at least one abovementioned cosmetic composition.

The present invention further relates to the use of a dispersion or of a composition according to the invention, for improving the surface appearance of the skin, more particularly for moisturizing the skin and/or reducing wrinkles and fine lines.

Throughout the description, the expression “comprising a” should be understood as synonymous with “comprising at least one”, unless otherwise specified. The expressions “comprised between . . . and . . . ”, “from . . . to . . . ” and “ranging from . . . to . . . ” are to be understood as including the boundaries, unless otherwise specified.

Particular examples of implementation of the method according to the invention for obtaining dispersions 10 will now be described.

### EXAMPLES

Unless otherwise indicated, in the following examples:
steps (iii) and (iv) are carried out by means of a microfluidic device as described in WO2015055748. The device is suitable for heating the fatty phase, or optionally, even the aqueous phase, to 80° C.
The suspension-ability is evaluated by means of the following stability test. A dispersion is then packaged in three 30 ml polypropylene (PP) receptacles filled to half. After 1 day at room temperature, each test undergoes one of three transport tests (one receptacle per test), namely:
   roller test (i.e. horizontal circular motion): Wheaton reference, for 1 hour;
   vibrating table (i.e. vertical circular movement): Heidolph Unimax reference 1010, for 1 hour; and
   3D mixer (i.e. random movements): for 6 minutes.
The flow threshold is evaluated using a rheometer shear rate sweep protocol (reference TA Instruments), according to the method described in H. A. Barnes, A Handbook of Elementary Rheology; Institute of Non-Newtonian Fluid Mechanics. University of Wales, 2000 or in http://www.tainstruments.com/pdf/literature/RH025.pdf.
The transparency of the gelled continuous aqueous phase is determined as follows: the composition to be tested is poured into a 30 ml Volga jar, the composition is left for 24 hours at ambient temperature and a white sheet is placed beneath, on which a cross approximately 2 mm thick is drawn with a black felt. If the cross is visible to the naked eye in daylight at an observation distance of 40 cm, the composition is transparent or translucent.

The stickiness, the play-time and the breakage-in-water are evaluated from a blind test on a panel of 24 women between 22 and 45 years old. Each woman applies 0.25 grams of the composition to be tested on a forearm.

The play-time can be defined as the application time of the tested composition, and in particular the time during which the user can apply the composition until full penetration.

The breakage-in-water can be defined as the ability of a composition to release or not release water upon application, and thus to impart a fresh and moisturizing effect. In others, it concerns the sensation felt when a gel breaks under the pressure applied and releases the water same contains.

### Scoring Criteria:

TABLE 1

| RATING CRITERIA | + | ++ | +++ |
|---|---|---|---|
| Suspension-ability of | Insufficient | Average suspension- | Satisfactory |
| drops in the gelled | suspension-ability | ability (duration | suspension-ability |
| aqueous phase* | (duration <1 week) | between 1 week and | (duration >1 |
| | | 1 month) | month) |
| Transparency of the | Opaque | Translucent | Transparent |
| gelled aqueous phase | | | |
| No stickiness upon | Marked stickiness | Moderately sticky | Weakly sticky |
| application | that stays after | passage that evolves | passage that |
| | application | to a braking finish | evolves to a soft |
| | | | finish |
| Play-time | Long application time | Average application | Fast application |
| | (i.e. >2 min) | time (i.e. between 1 | time |
| | | and 2 min) | (i.e. <1 min) |
| Breakage-in-water | Low breakage-in- | Medium breakage-in- | Satisfactory |
| | water (no | water (i.e. lower than | breakage-in-water |
| | transformative effect) | a carbomer gel | (i.e. similar to |
| | | (0.28%)) | carbomer gel |
| | | | (0.28%)) |

*evaluated at ambient temperature

### Example 1: Impact of the Hydrophilic Gelling Agent Apt to Gel in the Presence of At Least One Salt

In the present example, 3 dispersions are prepared using the abovementioned microfluidic manufacturing method wherein step (vi) is based on step (vi2).

The compositions of the starting phases are as follows [Table 2]:

| | | | 1A | 1B | 1C |
|---|---|---|---|---|---|
| | | | (comp.) | (inv.) | (inv.) |
| Phase | Raw materials | INCI | % | | |
| Fatty phase | Labrafac CC | Caprylic/Capric triglyceride | Q.S.* | Q.S.* | Q.S.* |
| (FP) | Nikkol | Limnanthes Alba | 15 | 15 | 15 |
| | Meadowfoam oil | (Meadowfoam) Seed Oil | | | |
| | EMC30 | Castor Oil/IPDI Copolymer | 33.33 | 33.33 | 33.33 |
| | | (and) Caprylic/Capric | | | |
| | | Triglyceride | | | |
| | Natpure Col RED | | 0.012 | 0.012 | 0.012 |
| | LC318L | | | | |
| Total | | | 100 | 100 | 100 |
| Aqueous | Osmosed water | Aqua | Q.S.* | Q.S.* | Q.S.* |
| phase (AP) | Microcare PE | Phenoxyethanol | 0.89 | 0.89 | 0.89 |
| | Microcare | Penylene glycol | 2.22 | 2.22 | 2.22 |
| | Emollient PTG | | | | |
| | Glycerin 4811 | Glycerin | 2.22 | 2.22 | 2.22 |
| | Zemea select | Propanediol | 3.22 | 3.22 | 3.22 |
| | Propanediol | | | | |
| | EDETA BD | Disodium EDTA | 0.044 | 0.044 | 0.044 |
| | Butylene glycol | Butylene glycol | 2.56 | 2.56 | 2.56 |
| | 1.3 | | | | |
| | Carbopol Ultrez | Carbomer | 0.28 | 0 | 0 |
| | 10 | | | | |
| | Satiagel VPC 508 | Carrageenan (and) | 0 | 0.4 | 0 |
| | P | Chondrus Crispus Extract | | | |
| | KELCOGEL CG- | Gellan gum | 0 | 0 | 0.21 |
| | LA | | | | |
| Total | | | 100 | 100 | 100 |
| Additional | Osmosed water | Aqua | Q.S.* | Q.S.* | Q.S.* |
| solution | NaOH | Sodium hydroxide | 0.34 | 0 | 0 |
| (BF) | Sodium chloride | SODIUM CHLORIDE | 0 | 10 | 2 |

| | | | |
|---|---|---|---|
| Total | 100 | 100 | 100 |

*Quantum satis.

The preparation of the FP, AP and BF falls within the general knowledge of a person skilled in the art. For carrying out steps (iii), (iv) and (vi2), the flow-rates are as follows:

TABLE 3

| Phase | Flow-rate per nozzle (ml/hr) |
|---|---|
| AP | 120 |
| FP | 16.28 |
| BF | 14.28 |

The monodisperse drops of dispersed fatty phase have a size of approximately 1200 μm.

The results are summarized in the following table.

TABLE 4

| | 1A | 1B | 1C |
|---|---|---|---|
| | (comp.) | (inv.) | (inv.) |
| Suspension-ability of drops | OK/5 | OK/15 | OK/5 |
| in the gelled aqueous | | | |
| phase/Flow threshold (in Pa) | | | |
| Transparency of the gelled aqueous phase | +++ | +++ | +++ |
| Absence of stickiness | +++ | +++ | ++ |
| Play-time | +++ | ++ | ++ |
| Breakage-in-water | +++ | +++ | +++ |

The example 1 shows that it is possible to manufacture macroscopic dispersions by means of a microfluidic method without resorting to the use of carbomer, by replacing the “carbomer/sodium hydroxide solution” by a “hydrophilic gelling agent apt to gel in the presence of at least one salt/salt solution” system.

Example 1 also shows that the dispersions according to the invention are stable and have satisfactory performances and at least similar to same of the prior art represented by the composition 1A.

### Example 2: Impact of the Content of Hydrophilic Gelling Agent Apt to Gel in the Presence of at Least One Salt

In the present example, 4 dispersions are prepared using the abovementioned microfluidic manufacturing method wherein step (vi) rests on step (vi2). The compositions of the starting phases are the following.

TABLE 5

| | | | 2A | 2B | 2C | 2D |
|---|---|---|---|---|---|---|
| | Raw | | (inv.) | (inv.) | (inv.) | (inv.) |
| Phase | materials | INCI | % | | | |
| Fatty phase | Identical to the fatty phase (FP) of example 1 | | | | | |
| (FP) | | | | | | |
| Aqueous | Osmosed | Aqua | Q.S.* | Q.S.* | Q.S.* | Q.S.* |
| phase (AP) | water | | | | | |
| | Microcare PE | Phenoxyethanol | 0.89 | 0.89 | 0.89 | 0.89 |
| | Microcare | Penylene glycol | 2.22 | 2.22 | 2.22 | 2.22 |
| | Emollient PTG | | | | | |
| | Glycerin 4811 | Glycerin | 8.89 | 8.89 | 8.89 | 8.89 |
| | Zemea select | Propanediol | 7.78 | 7.78 | 7.78 | 7.78 |
| | Propanediol | | | | | |
| | EDETA BD | Disodium EDTA | 0.044 | 0.044 | 0.044 | 0.044 |
| | Butylene | Butylene glycol | 5.56 | 5.56 | 5.56 | 5.56 |
| | glycol 1.3 | | | | | |
| | Satiagel VPC | Carrageenan (and) | 0.2 | 0.8 | 0 | 0 |
| | 508 P | Chondrus Crispus | | | | |
| | | Extract | | | | |
| | Kecolgel CG | Gellan gum | 0 | 0 | 0.08 | 0.125 |
| | LA | | | | | |
| Total | | | 100 | 100 | 100 | 100 |
| Additional | Osmosed | Aqua | Q.S.* | Q.S.* | Q.S.* | Q.S.* |
| solution | water | | | | | |
| (BF) | Sodium | SODIUM CHLORIDE | 10 | 10 | 2 | 2 |
| | chloride | | | | | |

| | | | | |
|---|---|---|---|---|
| Total | 100 | 100 | 100 | 100 |

The preparation of the FP, AP and BF falls within the general knowledge of a person skilled in the art. For carrying out steps (iii), (iv) and (vi2), the flow-rates are identical to same described in example 1.

The monodisperse drops of dispersed fatty phase have a size of approximately 1,200 μm.

The results are summarized in the following table.

TABLE 6

| | 2A | 2B | 2C | 2D |
|---|---|---|---|---|
| Processable by the microfluidic | ++ | ++ | ++ | ++ |
| method | | | | |
| Suspension-ability of drops in the | + | +++ | ++ | +++ |
| gelled aqueous phase | | | | |
| Transparency of the gelled aqueous | +++ | +++ | +++ | +++ |
| phase | | | | |
| Absence of stickiness | +++ | ++ | +++ | +++ |
| Play-time | +++ | ++ | +++ | +++ |
| Breakage-in-water | ++ | +++ | ++ | ++ |

Example 2 shows that the content of hydrophilic gelling agent apt to gel in the presence of at least one salt makes it possible to modulate the performances of the dispersions according to the invention.

### Example 3: Impact of the Content of Salt in the Additional Solution

In the present example, 6 dispersions are prepared using the abovementioned microfluidic manufacturing method wherein step (vi) rests on step (vi2). The compositions of the starting phases AP and FP and the flow-rates are identical to same described in example 1B hereinabove.

In example 3, the content of salt of the additional solution (BF) is varied in accordance with the table below.

TABLE 7

| Raw | | 3A | 3B | 3C | 3D | 3E | 3F |
|---|---|---|---|---|---|---|---|
| materials | INCI | % | | | | | |
| Osmosed water | Aqua | Q.S.* | | | | | |
| Sodium chloride | SODIUM CHLORIDE | 4 | 6 | 8 | 12 | 14 | 16 |

100

The monodisperse drops of dispersed fatty phase have a size of approximately 1,200 μm.

The results are summarized in the following table.

TABLE 8

| Criteria | 3A | 3B | 3C | 3D | 3E | 3F |
|---|---|---|---|---|---|---|
| Processable by the | + | ++ | +++ | +++ | ++ | + |
| microfluidic method | | | | | | |
| Suspension-ability of drops | + | + | ++ | +++ | ++ | ++ |
| in the gelled aqueous phase | | | | | | |
| Flow threshold(Pa) | 0.5 | 5 | 8.5 | 15.5 | 7.8 | 7 |
| Transparency of the | +++ | +++ | +++ | +++ | +++ | +++ |
| gelled aqueous phase | | | | | | |
| Absence of stickiness | +++ | +++ | +++ | +++ | +++ | +++ |
| Play-time | ++ | ++ | ++ | ++ | + | + |
| Breakage-in-water | + | + | ++ | +++ | ++ | ++ |

Example 3 shows that the salt content in the additional solution (BF) serves to modulate the properties of the dispersions according to the invention in terms of suspension-ability and, mechanically, of processability.

Surprisingly, the variation in the content of salt has little impact on the criterion of “absence of stickiness”.

### Example 4: Manufacture of a Dispersion According to the Invention

Example 4 differs from example 1B by the addition, in aqueous phase (AP), of an additional hydrophilic gelling agent different from a hydrophilic gelling agent apt to gel in the presence of at least one salt, namely preBIULIN C90 (INCI: Cellulose Gum (and) Xanthan Gum (and) Inulin (and) Cellulose (and) Glucose (and) Fructose) at 0.3% relative to the weight of the aqueous phase. Similar results were obtained by replacing preBIULIN C90 with Sucraclear HC-31 (INCI: Chondrus Crispus Powder (and) Cellulose Gum (and) Ceratonia Siliqua (Carob) Gum (and) Glucose) or Sucraclear V2 (INCI: Cellulose Gum, Chondrus Cripsus Powder (Carageenan), Ceratonia Siliqua Gum, glucose) at the same concentration.

The monodisperse drops of dispersed fatty phase have a size of approximately 1,200 μm.

The results are summarized in the following table.

TABLE 9

| Criteria | 1B | 4 |
|---|---|---|
| Processable by the microfluidic method | ++ | +++ |
| Suspension-ability of drops in the gelled aqueous phase | +++ | +++ |
| Transparency of the gelled aqueous phase | +++ | +++ |
| Absence of stickiness | +++ | ++ |
| Play-time | ++ | ++ |
| Breakage-in-water | +++ | +++ |

Example 4 shows that the presence of an additional hydrophilic gelling agent, in the present case Sucraclear V2 improves in particular the properties of a dispersion according to the invention in terms of microfluidic processability.

Without wishing to be bound by any theory, the applicant believes that the presence of an additional hydrophilic gelling agent increases the viscosity of the continuous aqueous phase without increasing the content of hydrophilic gelling agent apt to gel in the presence of at least one salt, which would have the consequence of degrading the feeling too significantly.

### Example 5: Manufacture of a Dispersion with a Method According to the Invention Comprising Step (vi1) and Step (vi2)

In the present example, a dispersion is prepared using the above-mentioned microfluidic manufacturing method wherein step (vi) comprises a step (vi1) and a step (vi2).

Example 5 differs from example 1B by the addition, in aqueous phase (AP), of 0.15% sodium chloride relative to the weight of the aqueous phase (AP).

The additional solution (BF) is thus adapted accordingly, as described hereinbelow.

TABLE 10

| Phase | Raw materials | INCI | % |
|---|---|---|---|
| Additional | Osmosed water | Aqua | Q.S.* |
| solution (BF) | Sodium chloride | SODIUM CHLORIDE | 8.65 |

| | |
|---|---|
| Total | 100 |

*Quantum satis.

The preparation of the FP, AP and BF falls within the general knowledge of a person skilled in the art.

The preparation of such mixture falls within the general knowledge of a person skilled in the art

The configuration of the flow-rates is as follows:

TABLE 11

| Phase | Flow-rate per nozzle (ml/hr) |
|---|---|
| AP | 120 |
| FP | 16.28 |
| BF | 14.28 |

The monodisperse drops of dispersed fatty phase have a size of approximately 1200 μm.

Compared with example 1B, example 5 demonstrates advantageous properties relating to the microfluidic method, mainly during the period between the formation of the dispersed phase drops and the injection of the additional solution.

Such advantage translates into a more stable and hence more robust manufacturing method, easier collection and a better-quality bulk due to an improved suspension of the drops of the aqueous phase, and thus a more effective prevention of the appearance of creaming or coalescence phenomena of the drops before the injection of the additional solution (BF).

1. A method of forming a dispersion comprising drops having a fatty phase, which drops are dispersed in a gelled continuous aqueous phase, the method comprising the steps of:
(i) providing a fatty phase comprising at least one oil and, optionally, at least one lipophilic gelling agent;
(ii) providing an aqueous phase, that is substantially immiscible with the fatty phase, comprising at least water and at least one hydrophilic gelling agent capable of gelling in the presence of at least one salt;
(iii) forming fatty phase drops in the aqueous phase or the gelled continuous aqueous phase;
(iv) conveying the drops in a circulation duct;
(v) recovering a dispersion comprising drops and the gelled continuous aqueous phase in a container; and
at least one of the steps of:
(vi1) before step, adding, to the aqueous phase, at least one portion of an aqueous solution; and/or
(vi2) injecting at least one portion of the aqueous solution into the circulation duct or at the outlet of the circulation conduit upstream of the container,
said aqueous solution comprising at least one salt apt to react with the hydrophilic gelling agent,
the dispersion being free of carbomer.

2. The method according to claim 1, wherein the preparation of the gelled continuous aqueous phase according to step comprises at least the following steps:
(a) adding the aqueous solution to the aqueous phase;
(b) returning the mixture obtained in step (a) to ambient temperature; and
(c) optionally, shearing the mixture obtained in step.

3. The method according to claim 1, wherein the drops having a diameter greater than or equal to 100 μm represent a volume greater than or equal to 60% of the total volume of the dispersed phase and/or at least 60% of the drops have a mean diameter greater than or equal to 100 μm.

4. The method according to claim 1, wherein the hydrophilic gelling agent apt to gel in the presence of at least one salt is a polyelectrolyte reactive to at least one salt.

5. The method according to claim 1, wherein the hydrophilic gelling agent apt to gel in the presence of at least one salt is chosen from natural polymers, biosynthetic polymers, modified polymers, and mixtures thereof.

6. The method according to claim 1, wherein the aqueous phase comprises between 0.01% and 5% by weight of hydrophilic gelling agent(s) to be gelled in the presence of at least one salt relative to the total weight of the aqueous phase.

7. The method according to claim 1, wherein the salt is monovalent, or multivalent.

8. The method according to claim 1, wherein the aqueous solution comprises between 0.01% and 30% by weight of salt(s) relative to the total weight of the aqueous solution.

9. The method according to claim 1 wherein the drops and the aqueous phase flow along a local axis in the circulation duct, the injection of the aqueous solution taking place substantially parallel to the local axis.

10. The method according to claim 1, wherein the injection of the aqueous solution is carried out at the outlet of the circulation duct.

11. The method according to claim 1, wherein same includes, upstream of the flow step, a step of forming drops in the circulation duct.

12. The method according to claim 1, wherein the dispersion comprised from 1% to 60% by weight of dispersed fatty phase relative to the total weight of the dispersion.

13. The method according to claim 1, wherein the lipophilic gelling agent is chosen from organic or inorganic, polymeric or molecular lipophilic gelling agents; fats which are solid at ambient temperature and pressure; and mixtures thereof.

14. The method according to claim 1, wherein the dispersion comprises from 0.5% to 30% by weight of lipophilic gelling agent(s) relative to the total weight of the fatty phase.

15. The method according to claim 1, wherein the aqueous phase and/or the aqueous solution further comprises at least one additional hydrophilic gelling agent different from a hydrophilic gelling agent apt to gel in the presence of at least one salt.

16. The method according to claim 1, wherein the dispersion does not comprise any surfactant.

17. An apparatus for forming a dispersion comprising drops, comprising:
a circulation duct containing drops of fatty phase in an aqueous phase substantially immiscible with the fatty phase;
a container for the recovery of a dispersion comprising drops and the aqueous phase;
a tank containing an aqueous solution comprising at least one salt;
at least one injection line for injecting the aqueous solution, coupled to the tank, and coming into the circulation duct or at the outlet of the circulation duct, upstream of the container,
the fatty phase comprises at least one oil and optionally at least one lipophilic gelling agent;
the aqueous phase, substantially immiscible with the fatty phase, comprises at least water and at least one hydrophilic gelling agent apt to gel in the presence of at least one salt; and
the dispersion being free of carbomer and, optionally, of amodimethicone.

18. The apparatus according to claim 17, characterized in that the circulation duct extends along a local axis of circulation of the drops and of the aqueous phase, the injection line coming out coaxially with the local axis.

19. A dispersion comprising drops of fatty phase dispersed in a continuous gelled aqueous phase, wherein:
the drops having a diameter greater than or equal to 100 μm represent a volume greater than or equal to 60% of the total volume of the dispersed phase and/or at least 60% of the drops have a mean diameter greater than or equal to 100 μm.
the fatty phase comprises at least one oil and optionally at least one lipophilic gelling agent; and
the gelled continuous aqueous phase comprises at least water, at least one hydrophilic gelling agent gelled by at least one salt,
the dispersion being free of carbomer and, optionally, of amodimethicone.
